(19) United States
(12) Patent Application Publication
(10) Pub. No.: US 2014/0257418 A1
(43) Pub. Date: Sep. 11, 2014

(54) SURGICAL TOOL HOLDER

(71) Applicant: KYPHON SARL, Neuchatel (CH)

(72) Inventor: Amy L. Arthur, Mountain View, CA (US)

(73) Assignee: KYPHON SARL, Neuchatel (CH)

(21) Appl. No.: 13/788,687

(22) Filed: Mar. 7, 2013

(51) Int. Cl. A61B 19/02

(52) U.S. Cl. CPC A61B 19/0256
USPC 606/86 R

(57) ABSTRACT

A device is provided for holding a surgical tool comprising a base extending along a longitudinal axis between a proximal end and a distal end. A retaining portion is disposed at the proximal end of the base configured to receive the surgical tool and hold the surgical tool at a fixed angle relative to the longitudinal axis. At least one opening extends through the distal end of the base configured to receive a fixation device to attach the base to a patient so as to stabilize the device while the surgical tool is manipulated. Methods of use are provided.

### TECHNICAL FIELD

The present disclosure relates to a surgical tool holder configured for use in the treatment of bone defects, such as, for example, bone fractures.

### BACKGROUND

Fractures, lesions and collapsing of bone structure can occur in humans due to age, disease or trauma. There are many areas of bone that are prone to collapsing/depression, such as vertebra, the proximal humerus, the tibial plateau, the distal radius and the calcaneus. A surgical tool, such as, for example, an inflatable bone tamp may be used to create voids for bone void filler (BVF) material. The IBT or a separate tool is then used to deliver BVF to a bone defect to restore collapsed bone and re-align bone fragments caused by fractures, for example. That is, the bone void filler may be used to fill any voids in the bone, such as, for example, fractures, after the inflatable bone tamp is removed. Precise positioning of the inflatable bone tamp adjacent to the bone defect is essential to properly restore the alignment of the bone. If the inflatable bone tamp is improperly placed, misalignment of the bone can occur. The anatomy of the patient often provides little assistance in maintaining the inflatable bone tamp in a position that places the inflatable bone tamp in a desired position adjacent the bone void, making it difficult to achieve proper placement of the inflatable bone tamp and to hold the tool for delivering BVF. This disclosure provides an improvement over prior art technologies.

### SUMMARY

This application is directed to a surgical tool holder for use in treating bone defects, such as, for example, fractures or lesions. In one embodiment, in accordance with the principles of the present disclosure, a device is provided for holding a surgical tool comprising a base extending along a longitudinal axis between a proximal end and a distal end. A retaining portion is disposed at the proximal end of the base configured to receive the surgical tool and hold the surgical tool at a fixed angle relative to the longitudinal axis. At least one opening extends through the distal end of the base configured to receive a fixation device to attach the base to a patient so as to stabilize the device while the surgical tool is manipulated.

In one embodiment, in accordance with the principles of the present disclosure, a device is provided for holding a surgical tool comprising a base extending along a longitudinal axis between a proximal end and a distal end. A retaining portion is disposed at the proximal end of the base configured to receive the surgical tool and hold the surgical tool at a fixed angle relative to the longitudinal axis. At least one through hole extends through the distal end of the base configured to receive a K-wire to attach the base to a patient and stabilize the device.

In one embodiment, in accordance with the principles of the present disclosure, a method is provided for holding a surgical tool is provided. The method includes providing a surgical tool holder comprising: a base extending along a longitudinal axis between a proximal end and a distal end, a retaining portion disposed at the proximal end of the base configured to receive the surgical tool and hold the surgical tool at a fixed angle relative to the longitudinal axis, and at least one opening disposed in the distal end of the base configured to receive a K-wire; positioning the surgical tool within the retaining portion such that the surgical tool is disposed at the fixed angle; and positioning the K-wire through the at least one opening to attach the base with the body of the patient and stabilize the device

### BRIEF DESCRIPTION OF THE DRAWINGS

The present disclosure will become more readily apparent from the specific description accompanied by the following drawings, in which:

FIG. 1 is a side, cross sectional view of one particular embodiment of a surgical tool holding device in accordance with the principles of the present disclosure; and

FIG. 2 is a side, cross sectional view of one particular embodiment of a surgical tool holding device in accordance with the principles of the present disclosure.

### DETAILED DESCRIPTION

Distal radius factures comprise a substantial portion of traumatic factures. Surgical treatment may involve reduction with metal bone tamps, elevators, fracture fixation with pins, external fixation or plate and screw implants. Inflatable bone tamps (IBT) are also utilized to repair distal radius fractures. An IBT is used percutaneously to reduce comminuted, articular depressions in a controlled manner. The IBT can also be used to reduce non-articular fractures or create well-defined voids for BVF. Fracture morphologies, such as, for example, “die-punch” fractures are especially suited for correction by an IBT. IBTs are deployed to a surgical site, such as, for example, a bone defect through a working cannula. IBTs create well-defined voids. After the bone cavity or void(s) is created by the IBT, the IBT is removed from the cannula and a material, such as, for example a bone void filler is delivered through the cannula and into the void or voids. The bone void filler may be used in conjunction with percutaneous pins, ex-fix, screws, or other implantable hardware for fracture fixation.

Percutaneous delivery of bone filler material through the cannula to bone voids in various parts of a patient's anatomy, such as, for example, a distal radius of the patient can be difficult. For example, if the distal end of the cannula is not open to the bone void of voids when the bone filler material is delivered, the bone void filler material can get stuck in the delivery tool. Furthermore, the distal end of the cannula must be positioned adjacent the bone void or voids to allow the bone void filler to be delivered through the cannula to the bone void or voids. There is often little soft tissue and/or bony anatomy to hold the cannula in a stable position during delivery of the bone filler material. To stabilize the cannula, a surgical assistant is often required to provide an extra pair of hands to hold the cannula while a surgeon delivers the bone filler material to the bone void or voids through the cannula. The present disclosure provides a device that acts as a second pair of hands to hold the cannula at a fixed trajectory to ensure that the cannula is properly positioned relative to the bone void or voids.

In one embodiment, the holding device includes a retaining portion for holding a cannula at a fixed trajectory and a base portion. The base portion includes pre-formed holes for K-wires. The holes are sized for clearance of the K-wires. In one embodiment, the holes are configured to provide an interference fit with a respective K-wire to fix the K-wire relative to the base. In one embodiment, the holes can be filled with a soft, deformable material, such as, for example, silicon rubber, to fix the K-wire relative to the base.

In one embodiment, the holes extend perpendicular to a longitudinal axis defined by the base. In one embodiment, the holes are at an oblique angle relative to the longitudinal axis defined by the base. The oblique angles of the holes may point distally or proximally. The distally pointed angles are positioned such that the holes guide the K-wire for provisional or terminal fixation.

In one embodiment, slots are provided in the base. The slots provide flexibility K-wire placement. That is, slots allow K-wires to be disposed at various angles relative to the longitudinal axis defined by the base, depending upon the preference of a medical practitioner. The slots may be empty or may be filled with a soft, deformable material, such as, for example, silicone rubber. The silicon provides a friction fit for a K-wire positioned in the slot. In one embodiment, the K-wires are driven into the base and have unicortical or bicortical purchase within bone. In one embodiment, the K-wires may extend into the ulna.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, distal and proximal, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament and/or bone, repairing a fracture or break in bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

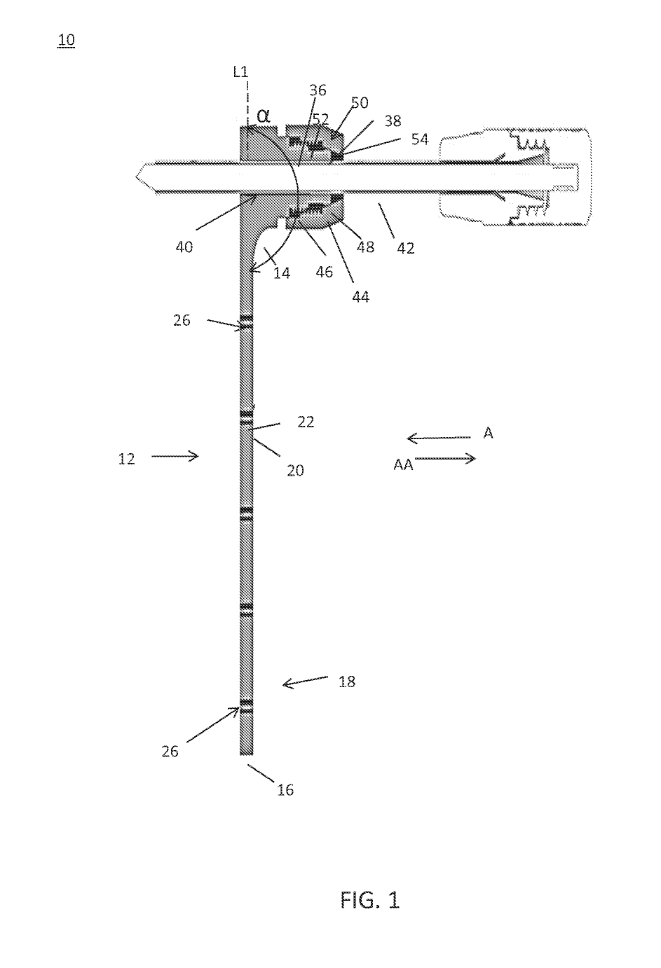

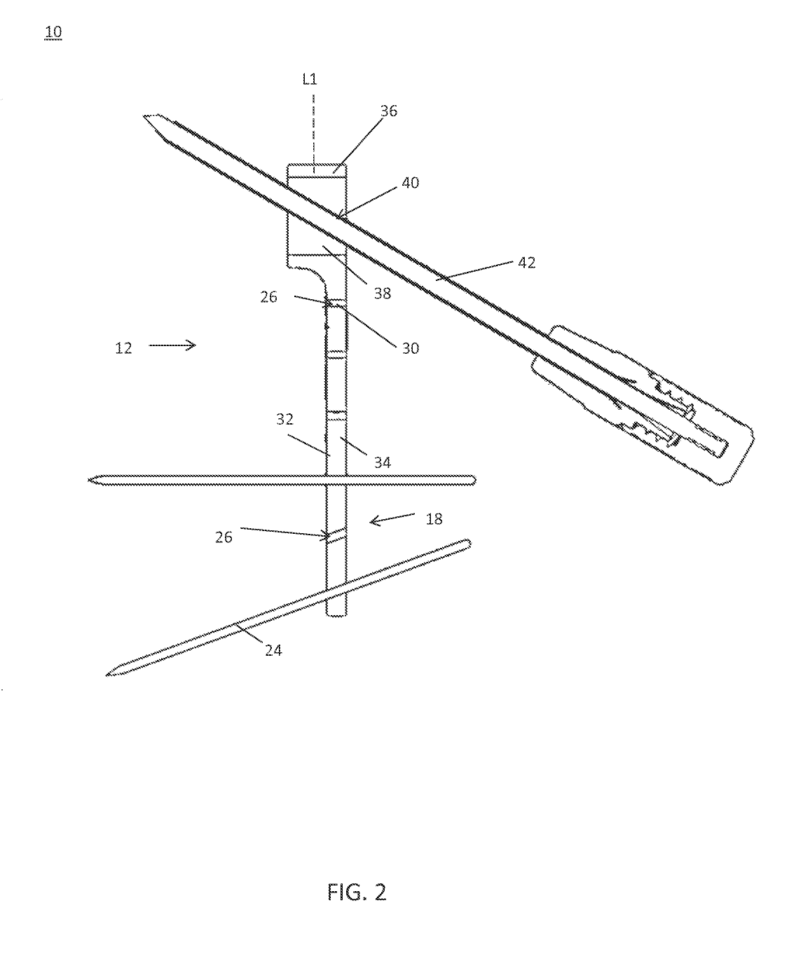

The following discussion includes a description of device for holding a surgical tool used for treating bone defects, such as, for example, bone lesions, fractures and/or collapsed bone and related methods of employing the device in accordance with the principles of the present disclosure. Alternate embodiments are also disclosed. Reference will now be made in detail to the exemplary embodiments of the present disclosure, which are illustrated in the accompanying figures. Turning now to FIGS. 1-2, there are illustrated components of the holding device for holding a surgical tool for treating bone defects in accordance with the principles of the present disclosure.

The components of the holding device can be fabricated from biologically acceptable materials suitable for medical apparatuses, including metals, synthetic polymers, ceramics, thermoplastic and polymeric material and/or their composites. For example, the components of the holding device, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan, Fe—Mn—Si and Fe—Ni—Co—Ti composites), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers based materials, polymeric rubbers, polyolefin rubbers, semi-rigid and rigid materials, thermoplastic elastomers, thermoset elastomers, elastomeric composites, rigid polymers including polyphenylene, polyamide, polyimide, polyetherimide, polyethylene, epoxy, and composites of metals and calcium-based ceramics, composites of PEEK and calcium based ceramics, and combinations of the above materials.

Various components of the holding device may have material composites, including the above materials, to achieve various desired characteristics such as strength, rigidity, elasticity, compliance, and biomechanical performance, durability and to provide a non-stick surface. The components of the holding device may be monolithically formed, extruded, coextruded, hot molded, cold molded, press molded, integrally connected or include fastening elements and/or coupling components, as described herein.

A surgical tool holding device 10, in accordance with the present disclosure, includes a base 12. Base 12 extends between a proximal end 14 and a distal end 16. Base 12 defines a longitudinal axis L1. Distal end 16 includes an engagement portion 18 configured for attaching device 10 to a body of a patient. In some embodiments, base 12 can include a surface that may be smooth, rough, textured, porous, semi-porous, dimpled and/or polished such that it facilitates engagement with the body. In some embodiments, the cross-section geometry of base 12 may have various configurations, such as, for example, round, oval, oblong, triangular, polygonal having planar or arcuate side portions, irregular, uniform or non-uniform.

Base 12 includes an inner surface 20 that defines at least one opening 22. Opening 22 is configured to receive a fixation device, such as, for example a K-wire 24. In one embodiment, opening 22 is configured as a through hole 26. Through hole 26 is configured to receive K-wire 24. It is envisioned that device 10 may include one or a plurality of holes 26 and a corresponding number of K-wires. In one embodiment, shown in FIG. 1, portion 18 includes a plurality of through holes 26 each extending perpendicular to axis L1. K-wires 24 fix device 10 to the anatomy of the patient.

In one embodiment, shown in FIG. 2, portion 18 includes a plurality of through holes 26 that are each angularly oriented with respect to axis L1. That is, the angularly oriented holes 26 extend at an acute angle relative to axis L1. One end of each angularly oriented through hole 26 is pointed distally with respect to the patient. The distally pointing position facilitates provisional and/or terminal fixation to the patient while the surgical tool is manipulated. An opposite end of each angularly oriented through hole 26 points proximally with respect to the patient. As shown in FIG. 2, portion 18 may include one or a plurality of holes 26 extending perpendicular to axis L1 and one or a plurality of holes 26 extending at an acute angle relative to axis L1.

In one embodiment, through holes 26 include a deformable material, such as, for example, a silicone insert 30 disposed therein. Insert 30 is configured to provide a friction fit with K-wire 24 to fix a respective K-wire 24 relative to base 12. In one embodiment, insert 30 comprises a resilient material such that insert 30 can deform to a second orientation and expand back to a first orientation. In some embodiments, all or only a portion of insert 30 may have a semi-rigid, rigid or elastic configuration, and/or have elastic properties, such as the elastic properties corresponding to the material examples described above, such that insert 30 provides a selective amount of expansion, contraction, collapse and/or extension. In some embodiments, insert 30 may be compressible in an axial direction. In one embodiment, insert 30 can include a plurality of separately attachable or connectable portions or sections, such as bands or loops, or may be monolithically formed as a single continuous element.

In one embodiment, opening 22 is a slot 32 configured to receive K-wire 24. It is envisioned that portion 18 may include one or a plurality of slots 32. Slots 32 are of a length configured to provide flexibility in placement of K-wire 24. That is, slots 32 each include a length extending along axis L1 that allows for angular orientation of K-wire 24 with respect to axis L1 or perpendicular orientation of K-wire 24 with respect to axis L1. K-wire 24 can be manipulated within slot 32 to provide the desired orientation and trajectory into the patient.

In one embodiment, slots 32 include a deformable material, such as, for example, a silicone insert 34 disposed therein. Insert 34 is configured to provide a friction fit with K-wire 24. In one embodiment, insert 34 comprises a resilient material such that insert 34 is deformable to a second orientation and expands back to a first orientation. In some embodiments, all or only a portion of insert 34 may have a semi-rigid, rigid or elastic configuration, and/or have elastic properties, such as the elastic properties corresponding to the material examples described above, such that insert 34 provides a selective amount of expansion, contraction, collapse and/or extension. In some embodiments, insert 34 may be compressible in an axial direction. Insert 34 can include a plurality of separately attachable or connectable portions or sections, such as bands or loops, or may be monolithically formed as a single continuous element.

In one embodiment, engagement portion 18 includes one or a plurality of through holes 26 and one or a plurality of slots 32. Providing base 12 with both holes 26 and slots 32 allows for greater options and precision when stabilizing device 10 to the patient.

Device 10 includes a retaining portion 36. Portion 36 includes an inner surface 38 that defines a passageway 40. Passageway 40 is configured to receive a surgical tool, such as, for example, a cannula 42. In some embodiments, inner surface 38 may be rough, textured, porous, semi-porous, dimpled and/or polished to facilitate engagement with cannula 42. Passageway 40 is disposed at a fixed angular orientation a relative to axis L1. Angle a ranges from 0-180 degrees with respect to axis L1 such that it provides a fixed trajectory into the body.

In one embodiment, shown in FIG. 1, portion 36 includes a collet 44 extending perpendicular to axis L1 having a first end 46 with a threaded outer surface and a resilient second end 48. Device 10 includes a cap 50 having a first end 52 with a threaded inner surface and a second end 54 with an angled inner surface. The threaded inner surface of end 52 is configured to engage the threaded outer surface of end 46. As the threaded inner surface of end 52 engages the threaded outer surface of end 46, cap 50 translates relative to base 12 in the direction shown by arrow A. As cap 50 translates in the direction shown by arrow A, the angled inner surface of end 54 engages the outer surface of end 48 causing the inner surface of end 48 to engage cannula 42. As the inner surface of end 48 engages cannula 42, cannula 42 becomes fixed relative to portion 36 so as to fix cannula 42 at a fixed angle relative to axis L1. That is, engaging the inner surface of end 48 with cannula 42 prevents cannula 42 from moving in the direction shown by arrow A or the direction shown by arrow AA.

In operation, assembly and use, device 10 is employed with a surgical procedure for treatment of a disorder affecting a bone, such as, for example a fracture in a vertebra or other bone of a patient. Device 10 is attached to the anatomy of the patient such that a distal end of cannula 42 is positioned adjacent the bone disorder or defect. K-wires 24 are positioned within through holes 26 and/or slots 32 at a desired angular orientation with respect to axis L1 to fix device 10 relative to the bone disorder or defect. Once device 10 is stabilized, cannula 42 is movable in the direction shown by arrow A or the direction shown by arrow AA within passageway 40.

In embodiments in which device 10 includes collet 44, the threaded inner surface of end 52 may be moved to engage the threaded outer surface of end 46 such that cap 50 translates relative to base 12 in the direction shown by arrow A until the inner surface of end 48 engages cannula 42 to prevent cannula 42 from moving in the direction shown by arrow A or the direction shown by arrow AA.

An inflatable bone tamp may be delivered through cannula 42 to a location adjacent the bone disorder or defect. The inflatable bone tamp may then be expanded to create a cavity or void in the bone. After the cavity or void is formed in the bone, the inflatable bone tamp is removed from device 10.

Bone filler material is then delivered through cannula 42 and into the cavity or void so as to at least partially fill the cavity or void and realign fragments of the fracture and/or elevate collapsed bone to its proper height. Device 10 maintains cannula 42 in a stabilized orientation with respect to the body such that a single practitioner can administer the bone filling material in a steady manner.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A device for holding a surgical tool comprising:
a base extending along a longitudinal axis between a proximal end and a distal end;
a retaining portion disposed at the proximal end of the base configured to receive the surgical tool and hold the surgical tool at a fixed angle relative to the longitudinal axis; and
at least one opening extending through the distal end of the base configured to receive a fixation device to attach the base to a patient so as to stabilize the device while the surgical tool is manipulated.

2. A device as recited in claim 1, wherein the at least one opening is a through hole configured to receive the fixation device.

3. A device as recited in claim 1, wherein the at least one opening is a slot configured to receive the fixation device.

4. A device as recited in claim 1, wherein the at least one opening extends perpendicular to the longitudinal axis of the base.

5. A device as recited in claim 1, wherein the at least one opening is angularly oriented with respect to the longitudinal axis.

6. A device as recited in claim 1, wherein the at least one opening is angularly oriented with respect to the longitudinal axis such that one end of the at least one opening points distally with respect to the patient thereby facilitating provisional or terminal fixation to the patient while the surgical tool is manipulated.

7. A device as recited in claim 1, wherein the at least one opening is angularly oriented with respect to the longitudinal axis such that one end of the at least one opening points proximally with respect to the patient.

8. A device as recited in claim 1, wherein the distal end of the base comprises at least one through hole and at least one slot.

9. A device as recited in claim 8, wherein at least one through hole is disposed at an acute angle relative to the longitudinal axis.

10. A device as recited in claim 8, wherein at least one through hole is disposed at an acute angle relative to the longitudinal axis.

11. A device as recited in claim 1, wherein the at least one opening includes a deformable material disposed therein so as to provide a friction fit with the fixation device.

12. A device as set forth in claim 1, wherein the fixation device is a K-wire.

13. A device as set forth in claim 1, wherein the surgical tool is a cannula.

14. A device for holding a surgical tool comprising:
a base extending along a longitudinal axis between a proximal end and a distal end;
a retaining portion disposed at the proximal end of the base configured to receive the surgical tool and hold the surgical tool at a fixed angle relative to the longitudinal axis; and
at least one through hole extending through the distal end of the base configured to receive a K-wire to attach the base to a patient and stabilize the device.

15. A device as recited in claim 14, wherein the distal end of the base includes at least one slot configured to receive the K-wire.

16. A device as recited in claims 14, wherein the at least one through hole extends perpendicular to the longitudinal axis.

17. A device as recited in claim 14, wherein the at least one through hole extends through the base at an acute angle relative to the longitudinal axis.

18. A device as recited in claim 14, wherein the at least one through hole includes a deformable material disposed therein to provide a friction fit with the K-wire.

19. A device as recited in claim 15, wherein the at least one slot includes a deformable material disposed therein to provide a friction fit with the K-wire.

20. A method for holding a surgical tool comprising:
providing a surgical tool holder comprising:
a base extending along a longitudinal axis between a proximal end and a distal end,
a retaining portion disposed at the proximal end of the base configured to receive the surgical tool and hold the surgical tool at a fixed angle relative to the longitudinal axis, and
at least one opening disposed in the distal end of the base configured to receive a K-wire;
positioning the surgical tool within the retaining portion such that the surgical tool is disposed at the fixed angle; and
positioning the K-wire through the at least one opening to attach the base with the body of the patient and stabilize the device.
